(19) United States
(12) Patent Application Publication
(10) Pub. No.: US 2023/0038914 A1
(43) Pub. Date: Feb. 9, 2023

(54) FENESTRATED BONE ANCHOR

(71) Applicant: SpineWelding AG, Schlieren (CH)

(72) Inventors: Andrea Müller, Winterthur (CH); Jörg Mayer, Niederlenz (CH); Domenico Romeo, Wohlen (CH)

(21) Appl. No.: 17/961,081

(22) Filed: Oct. 6, 2022

Related U.S. Application Data
(63) Continuation of application No. PCT/EP2021/059113, filed on Apr. 7, 2021.
Application No. PCT/EP2021/059113 is a continuation of application No. PCT/EP2021/059110, filed on Apr. 7, 2021.
Application No. PCT/EP2021/059110 is a continuation of application No. PCT/EP2021/059112, filed on Apr. 7, 2021.

(30) Foreign Application Priority Data
Apr. 8, 2020 (CH) 00427/20
Apr. 8, 2020 (CH) 00428/20
Apr. 8, 2020 (CH) 00429/20

(51) Int. Cl. A61B 17/86

(52) U.S. Cl. CPC A61B 17/86; A61B 2017/00955

(57) ABSTRACT

Disclosed is a fenestrated bone anchor suitable for being anchored in live bone tissue of a human or animal patient. The bone anchor comprises a shaft with a proximal end and a distal end and a circumferential surface extending from the proximal end to the distal end. The shaft further comprises a longitudinal cavity extending from the proximal end towards the distal end and a plurality of lateral channels extending through a wall of the shaft from the axial cavity to the circumferential surface. The lateral channel has a pear-shaped cross section and/or the thickness of the wall increases gradually in selected ones of directions towards the lateral channels. The fenestrated bone anchor is e.g. a component of a surgical system comprising an interbody fusion device of the stand-alone type.

### FIELD OF THE INVENTION

The invention is in the field of medical technology. It relates to a fenestrated bone anchor, i.e. to a surgical item being suitable for being anchored in live bone tissue of a human or animal patient and comprising a longitudinal cavity extending along an anchor axis from a proximal anchor end towards a distal anchor end, and at least one lateral channel connecting a circumferential anchor surface with the longitudinal cavity.

It also relates to a bone implant, in particular to a load-bearing bone implant suitable for being implanted in a human or animal patient between surfaces of live bones or bone fragments, suitable for bearing at least temporarily loads acting between these bones or bone fragments, and suitable for being integrated between the bones or bone fragments by bone growth after surgery. Such bone implants are applicable in surgical procedures such as e.g. spinal fusion, arthrodesis and osteotomy.

It further relates to a surgical system comprising an implant and at least one bone anchor suitable for fixating the implant relative to bone tissue in a human or animal patient, wherein the implant comprises a through opening and the bone anchor comprises a head and a shaft, wherein the implant and the bone anchor are adapted to each other for the anchor shaft to be able to pass through the through opening and the anchor head to be retainable relative to the through opening in a final position. The invention further relates to the bone anchor and the implant of the system.

### BACKGROUND OF THE INVENTION

Fenestrated bone anchors are widely known in the field of human and veterinary surgery. They are suitable in particular for an anchor retention with the aid of a bone cement or a material having thermoplastic properties, wherein the bone cement is pressed in a flowable state into the longitudinal cavity and from there through the lateral channel or channels to contact and possibly augment live bone tissue surrounding the anchor, or wherein the material having thermoplastic properties is positioned in the longitudinal cavity in a solid state, is liquefied therein by application of energy, for example vibration energy, and, in a liquefied state, is pressed through the lateral channel or channels for achieving a similar effect as achieved with the bone cement. Fenestrated bone anchors can also be used for administering pharmaceutical substances to the bone tissue surrounding the anchor.

Fenestrated bone anchors comprise a shaft and possibly a head. The circumferential shaft surface may comprise any sort of per se known means for retaining the anchor in bone tissue in addition to the possible retention achievable by the above described augmentation. Such retaining means are e.g. threads, teeth, cutting edges, ribs, resilient projections or also osseointegration enhancing surface roughness or surface coatings.

Fenestrated bone anchors are applicable e.g. for stabilizing bone fragments or misaligned bones relative to each other or for fixating soft tissue on bone tissue. Furthermore, they are applicable for fixating artificial items relative to bone tissue, such as e.g. bone plates, interbody fusion devices, intramedullary nails or rods, instrumentation for spine correction or support, and also sutures and cables for various applications. They are also applicable for bone augmentation. Bone anchors to be used in the named or other exemplary applications are simple fixation pins or screws with or without heads, pedicle screws, interference devices, suture anchors, dental implants etc.

EP 3 616 634 concerns screw implants for bone fusion. The screw implants may have a shaft with biomaterial windows into which biomaterial, such as bone graft can be filled. In one embodiment, the biomaterial windows can be in fluid communication with a cannulated opening that serves for receiving a guide wire or other type of instrument to assist in implantation. The diameter of the cannulation is smaller than a width of the biomaterial windows and does not serve for, and would not be suitable for, feeding the biomaterial windows with material. The biomaterial windows have shapes that are elongate, with long sides running parallel to the longitudinal axis between distal and proximal end sections.

The publication WO2010/096942 discloses a system with a plurality of fenestrated bone anchors. In addition to the plurality of fenestrated bone anchors, the disclosed system comprises an interbody fusion device of the stand-alone type, i.e., an interbody implant to be positioned between neighboring vertebral bodies and a plate suitable for retaining the implant in its position. The plate has a plurality of through openings and the bone anchors are adapted to the through openings and suitable for fixating the plate to the neighboring vertebral bodies. The implant and the plate may be two separate items, may be fixed to each other or may form together one integral part. The disclosed bone anchors have the form of fenestrated headed pins. They comprise a longitudinal cavity and lateral channels connecting the longitudinal cavity with the circumferential surface of the pin and they are described to be suitable for being anchored in the bone tissue of the vertebral bodies with the aid of a material having thermoplastic properties. For the anchoring process, an opening is provided in the bone tissue, the fenestrated pin is introduced into the opening, an element of the material having thermoplastic properties is inserted into the longitudinal cavity, energy, in particular vibration energy (in particular ultrasonic vibration energy) is applied to the thermoplastic element for liquefying the material having thermoplastic properties, and simultaneously with the application of the energy, the element of the material having thermoplastic properties is pressed into the cavity for displacing the liquefied material through the lateral channels into the adjacent bone tissue or between the circumferential pin surface and the adjacent bone tissue. For achieving preferred anchorage in the vertebral body, the lateral channels may be situated nearer to the proximal pin end than to the distal pin end (e.g. in the proximal half or in the most proximal third of the pin length) for deposition of the material having thermoplastic properties as near as possible to the cortical bone of the wall of the vertebral body, where the bone tissue has a stronger consistency for being penetrated by the liquefied material than the bone tissue nearer the center of the vertebral body.

The disclosure of the publication WO2010/096942 is enclosed herein by reference in its entirety.

### SUMMARY OF THE INVENTION

It is a first object of the present invention to provide an improved fenestrated bone anchor which has features as described above and which is particularly suitable for the above-mentioned anchorage with the aid of a material having thermoplastic properties, but is applicable for other applications also. The improvement of the fenestrated bone anchor regards in particular the at least one lateral channel, the material flow therethrough and its mechanical effect on the stability of the anchor.

It is another object of the present invention to provide a system comprising an implant with at least one through opening and at least one bone anchor with a head and a shaft, wherein the through opening and the anchor are adapted to each other for the anchor shaft to be able to pass through the through opening and the anchor head to be retained by a proximal surface of the implant or in the through opening, thereby limiting movement of the anchor in a distal direction and defining a final position of the anchor relative to the implant. The system further comprises a locking mechanism for locking the anchor in the named final position for preventing it from moving in a proximal direction (backing out) away from its final position. The locking mechanism of the system is to be operable without the necessity of a separate locking element and without a separate locking step, and it is to be manufacturable without a separate manufacturing step. Furthermore, the locking mechanism is to limit design and material choice of the implant or the bone anchor as little as possible. Preferable manufacturing processes suitable for manufacturing implant and/or bone anchor together with the locking mechanism are in particular additive manufacturing processes (3D-printing processes) as well as processes involving a material removal step, such as milling.

It is an even further object of the present invention to provide a load-bearing bone implant suitable for being implanted in a human or animal patient between surfaces of live bones or bone fragments, suitable for bearing at least temporarily loads acting between these bones or bone fragments, and suitable for being integrated between the bones or bone fragments by bone growth after surgery, wherein the bone implant is to enable more complete and more rapid bone ingrowth into the implant than know such implants, such that the bone tissue grown after surgery and in particular already during a rehabilitation period, can bear at least a relevant part of or even the full load acting on the bones between which it is implanted, or such that the implant can be designed with a minimum of material and virtually without regard for long term functionality (limited e.g. by material fatigue or resorbability of the used material) respectively. According to a first aspect of the invention, the fenestrated bone anchor achieving the above object comprises a shaft and possibly a head, the shaft having a proximal end, a distal end, and a longitudinal axis and a circumferential surface extending from the proximal end to the distal end, as well as a longitudinal cavity extending in the direction of the axis, if applicable, through the head and from the proximal shaft end towards the distal shaft end. Furthermore, the shaft comprises at least one lateral channel (e.g. two to four lateral channels) extending through a wall of the shaft from the longitudinal cavity to the circumferential surface. This wall has a wall thickness and the lateral channel has a cross section with an axial length and a width smaller than the axial length, wherein the width of the cross section of the lateral channel increases in a distal direction and/or the wall thickness increases gradually in selected ones of directions towards the lateral channel. This means that the cross section of the lateral channel or channels has a pear-shape and that in addition or alternatively the wall between the axial cavity and the circumferential surface has a thickness which is not the same around the longitudinal cavity but is largest at least partially where adjoining the lateral channel and is decreasing gradually in a direction away from the lateral channel.

The named design features make the bone anchor particularly suitable for an anchoring process by augmentation with the aid of a material having thermoplastic properties as described briefly further above, for applications in which more proximal augmentation is desired, and for applications in which the proximal anchor end is more rigidly fixated (e.g. in a plate or in cortical bone) than the distal anchor end (e.g. in trabecular bone) and therewith bending load increases from the distal end towards the proximal end. The named mechanical conditions are in particular applicable for anchors used for fixating e.g. a plate on a vertebral body or in a meta- or epiphyseal region of a long bone or also for suture anchors, in which cases the preferred anchorage is as near as possible to the cortex where bone density is highest and where a body of augmenting material can lean on the cortex and so to speak extend the thickness of the cortex.

The lateral channel may have a substantial axial extension of less than 30%, for example less than 25%, for example between 12% and 20% of a length of the bone anchor.

For many embodiments, the lateral channel needs to have a substantial axial extension (length) so that the bone tissue can be homogeneously infiltrated in a large range even if there is substantial quenching behavior, as is for example the case for thermoplastic material when it infiltrates the bone in a liquid state. An infiltration in a large area is advantageous not only for solid anchoring but also for reinforcing the bone tissue. In particular, the design measures of the pear-shaped cross section of the lateral channel and/or the wall thickness increasing towards the lateral channel make it possible to give the lateral channels a cross section guaranteeing satisfactory flow of the liquefied material even when the latter is subject to quenching and still giving the anchor mechanical characteristics which allows it to bear the bending stress even if the lateral channel is arranged in a proximal portion of the anchor. The named design measures result in the anchor being homogeneously stressed over its length at a minimum of notch stress, which is particularly important for anchors subjected to cyclical stressing.

Especially, the shape of the lateral channel may be chosen so that across the length (axial extension) of the lateral channel, the medium stress (comparison stress) is essentially homogeneous along the lateral channel. This allows to the lateral channel to have a substantial axial extension while the load-bearing strength is not substantially reduced. Especially, the ratio of the comparison stress to the material strength, when a certain bending moment is applied upon the bone anchor, may be designed to be essentially constant across the length of the lateral channel. To achieve this, the bending stiffness may be chosen to gradually decrease towards distally. This feature of the bending stiffness increasing towards proximally may be present across a substantial portion of the length of the lateral channel (for example in a middle region excluding a proximal and a distal end, for example across at least 40% or at least 50% of its length for example across at least 40% or at least 50% thereof) and not for example only at the proximal/distal end.

For example, the anchor may be designed for the stress at the rim of the lateral channel (given a certain applied bending moment) to vary along the length of the lateral channel by less than about 30% or less than about 20% or less than about 10% (defined as the difference between the maximum stress and the minimum stress divided by the average stress along the complete length), for example over half of the length of the lateral channel is situated and for example over the entire length of the lateral channel.

A possible design criterion may be that the so-called polar second moment of area J with respect to the longitudinal axis increases, for example continuously increases, towards proximally across the anchor portion in which the at least one lateral channel is situated, for example for at least a substantial portion of its length (for example in a middle region excluding a proximal and a distal end, for example across at least 40% or at least 50% of its length). Especially, the polar second moment of area J may be such that the value x*J is approximately constant across the anchor portion in which the at least one lateral channel is situated, where x is the distance to the proximal end of the bone anchor. In this, “approximately constant” may be defined as varying by not more than 20% or not more than 10% or even not more than 5%.

Fulfilling this design criterion may be helpful in view of the facts that in use the bone anchor is held proximally by relatively stiff structures, namely the implant (for example bone plate) and the cortical bone tissue, and that the lateral channel may be located at a relatively proximal position, away from the distal end.

A further, possibly additional design criterion may be that the material of the bone anchor, the strength of which according to the above design criterion relating to the second moment of area J generally increases towards proximally, is distributed in a manner that the tress along the rim of the lateral channel does not become too high compared to the stress elsewhere and is relatively homogeneous, as mentioned above.

To this end, one possibility is that the wall thickness increases towards the lateral channel. In addition or as an alternative, the shape of the lateral channel (or, to be precise its mouth in the outer surface of the bone anchor shaft) may have the mentioned pear shape. In this latter way, the stress in the more critical more proximal regions can be somewhat lowered along the rim of the lateral channel in that the stress is more distributed and the regions between the lateral channels take up more stress. For the shape of the mouth of the lateral channel, this means that, in addition or as an alternative to the wall thickness increasing towards the lateral channel, one or more of the following may hold:
At axial positions that correspond to axial positions of a middle region of the lateral channel, the circumferential width decreases continuously in a proximal direction. The circumferential width may for example decrease along at least 40%, at least 50% or at least 60% of the axial extension (from distal to proximal) of the lateral channel, and/or it may decrease from distal to proximal by at least 5%, at least 10% or at least 15%
At axial positions that correspond to axial positions of a middle region of the lateral channel, the polar second moment of area J increases continuously in a proximal direction. The polar second moment of area J may for example increase from distal to proximal along at least 30%, 40%, at least 50% or at least 60% of the axial extension of the lateral channel, for example by at least 5%, at least 10%, or at least 15% or 20% and for example at most 50%.
An average circumferential width of a proximal half of the lateral channel is substantially smaller than an average circumferential width of a distal half of the lateral channel. “Substantially smaller” in this context may mean smaller by at least 5% or by at least 10% or even by at least 20%.
The sidewalls are not parallel to each other but may approximate towards proximal, not only in a proximal and distal end region, but along a substantial portion of the lateral channel's axial extension. Especially, the sidewalls may be not parallel across at least 40%, at least 50% or at least 60% of the axial extension of the lateral channel.
The sidewalls of the lateral channel are not parallel to the longitudinal axis but extend essentially straight at an angle of between 1° and 20°, especially between 2° and 10°, in particular between 3° and 8°, for example about 5° to the axis, approximating towards proximal.
The sidewalls are not parallel to each other in a middle region located in the middle between the proximal end and the distal end of the lateral channel, but the sidewalls may approximate towards proximal.

The longitudinal cavity serves for inserting the material that is pressed into surrounding tissue through the at least one lateral channel. It may be essentially cylindrical, at least distally of the head and proximally of the distal end, for example circularly cylindrical (i.e. circular in cross section). A diameter or average diameter of the longitudinal cavity will be larger than a width of the lateral channel, for example substantially larger than the latter. The average diameter of the longitudinal cavity and/or the diameter of the longitudinal cavity at the position of the lateral channel may for example be between 1.1 times and 3 times the maximum width of the lateral channel (i.e., between 1.1 times and 3 times the circumferential width at the position where it is widest, which may be the case generally towards the distal end in embodiments in which the lateral channel is pear shaped). Especially, it may be between 1.2 times or 1.3 times and 2.5 times or between 1.4 times and 2 times, especially between 1.5 times and 1.9 times and for example about 1.7 times the maximum width.

A typical diameter of the longitudinal cavity may be between 1.5 mm and 4.5 mm, especially between 1.7 mm and 3.7 mm, for example about 3-3.2 mm or between 1.7 mm and 2.2 mm (the latter for cervical applications). More in general, the diameter of the longitudinal cavity may amount to between 40% and 87%, especially between 50% and 82% or between 60% and 80%, for example around 75%-79% of the bone anchor shaft diameter.

A height (axial extension of the lateral channel may be between 1.2 times and 4 times its maximum width, especially between 1.4 times and 3.2 times or between 1.6 times and 2.6 times, for example about 1.8-2.4 times the maximum width.

The distal end of the longitudinal cavity in the bone anchor may be closed. In a variant, it may have comparably small (i.e. radial extension being much smaller than the radial extension of the longitudinal cavity) axial opening for a guide wire or similar. The distal end may have a shape different from circularly symmetrical about the axis of the bone anchor. Rather, the channels may have an inner portion at radial positions within a radius of the longitudinal cavity in addition to having an outer portion formed by the (pear-shaped) exit opening in the wall around the cavity. Thus, the distal end of the longitudinal cavity may be angularly structured so as to direct different portions of the material to the different exit openings.

Such angular structure of the distal end of the longitudinal cavity may especially be advantageous in case the bone tissue around the bone anchor is not homogeneous and/or not isotropic: the structure of the distal cavity end ensures that also then the material is pressed out through the different exit opening in approximately equal amounts and not for example only through the exit opening where the resistance is smaller compared to the other exit openings. Especially if the material is thermoplastic material and is positioned in the longitudinal cavity in a solid state, it will have little possibility to evade the hydrostatic pressure by flowing back towards proximally and out of the opening where the resistance is the lowest. This is because the material is predominantly liquefied in contact with the distal end of the cavity and prevented from flowing back therefrom by the remaining thermoplastic material being pressed towards distally during the process. In this way the structures of the distal end serve for directing the material to the respective exit opening.

For example, the structures of the distal end comprise may comprise directing walls that extend radially between the inner portions of the channels.

The bone anchor consists of a medically acceptable metal, polymer of ceramic material and it may be manufactured using an additive manufacturing method. The bone anchor consists e.g. of a suitable titanium alloy, a CoCr-alloy or a molybdenum-rhenium alloy and is manufactured using a selective laser sintering a process, a selective laser melting process or an electron beam melting process.

The present invention also concerns a set of a fenestrated bone anchor as described in the present text together with a thermoplastic element in a solid state, the thermoplastic element having a shape adapted to be inserted into the longitudinal cavity from its proximal end and capable of being liquefied by being pressed against the distal end of the cavity while energy, especially mechanical vibration energy, is coupled into the thermoplastic element.

Such set may further comprise a sonotrode shaped to couple the mechanical vibration energy into the thermoplastic element. The sonotrode may especially have a distal end adapted to be inserted into the longitudinal cavity from its proximal end so as to liquefy a final portion of the thermoplastic element. In addition or as an alternative to the sonotrode, the set may have a guiding sleeve or guiding tube, the guiding sleeve or guiding tube adapted to be coupled to the bone anchor so as to guide the thermoplastic element upon insertion into the longitudinal cavity and/or within the longitudinal cavity.

An exemplary embodiment of the bone anchor which is made of a titanium alloy and is suitable for fixating a lumbar spinal fusion device as disclosed in the above-mentioned publication WO2010/096942 has about the following dimensions: overall length (head and shaft) 25 mm, axial position of the lateral channels 3 to 4 mm below the plate, axial length, distal width and proximal width of cross section of lateral channels 3.8 mm, 1.8 mm, 1.4 mm, wall thickness of shaft below head and in the area of the lateral channels 0.7 mm and 0.6 mm.

In embodiments, the bone anchor is part of a system constituting an interbody fusion device of the stand-alone type comprising an interbody fusion implant, a bone plate with a plurality of through openings and a plurality of bone anchors adapted to the through openings of the plate and suitable for fixating the plate relative to two neighboring vertebral bodies. Known such systems are e.g. disclosed in the publication WO2010/096942, which is enclosed herein in its entirety by reference. The plate of the system may constitute a fully separate element, may be fixed on the interbody implant or may form one integral part therewith. The interbody fusion implant is positioned between two neighboring vertebral bodies, the plate is arranged on the anterior or on a lateral side of the two vertebral bodies and is fixated thereto with the aid of the bone anchors.

Bone anchors that may be used in the system are elongate fenestrated bone anchors with a head and a shaft, which bone anchors are driven into the bone tissue of the vertebral body through the through openings of the plate, and are anchored in the vertebral body e.g. with the aid of a material having thermoplastic properties and being liquefied by application of e.g. vibration energy within a central cavity of the fenestrated anchor and pressed to the outside of the anchor through lateral channels to re-solidify for example within the trabecular structure of the bone tissue surrounding the anchor or between the bone tissue and the anchor.

According to a second aspect of the present invention, a surgical system is provided, the system comprising the anchor and an implant with a through opening adapted to the anchor. The anchor may be an anchor according to the first aspect or may be an anchor not according to the first aspect.

The surgical system according to the second aspect comprises an implant with at least one through opening defining an opening axis and at least one bone anchor with a head and a shaft and an anchor axis, the through opening and the bone anchor being adapted to each other for the anchor shaft to be able to pass through the through opening and the anchor head to be retained by a proximal surface of the implant or in the through opening, for example in a shallow indentation in the proximally facing face of the implant, the shallow indentation belonging to the through opening, in a final position. For locking the anchor in said final position, the system further comprises at least one locking element being moveable between a relaxed position in which it protrudes from a general level of a surface portion of the anchor or the through opening and a resiliently tensioned position in which it protrudes less from or is substantially flush with said level. Therein the locking element is an integral part of the bone anchor or of the implant, constituting a part of the named surface portion and of a bulk of the anchor or the implant situated underneath this surface portion. A void delimits the locking element in the surface portion and further extends underneath the locking element or through the named bulk.

The locking element may be formed as a resilient cantilever or resilient bending beam having a length and a width.

The locking element may further comprise a ramp and a locking surface being arranged adjacent to each other.

The length of the cantilever or bending beam may extend substantially parallel to the anchor axis or to the opening axis and the ramp and the locking surface are arranged adjacent to each other over the length of the cantilever or bending beam.

The locking element may be arranged on the shaft of the bone anchor and/or at least one of the through openings may comprise an undercut.

The locking element may be arranged on the shaft of the bone anchor.

The length of the cantilever or bending beam may extend substantially perpendicular to the anchor axis or the opening axis and the ramp and the locking surface may be arranged adjacent to each other over the width of the cantilever or bending beam.

The cantilever or bending beam may be arc shaped. A radius of curvature of the cantilever or bending beam may optionally correspond to a radial distance between the bending beam and the anchor axis or the opening axis.

The cantilever or bending beam may extend in a plane perpendicular to the anchor axis.

The cantilever or bending beam may be supported on both sides and may be integral with further material of the bone anchor on both sides.

The system may comprise a locking protrusion protruding radially from the cantilever or bending beam, the locking protrusion having the ramp and having a locking surface situated proximally of the ramp.

The cantilever or bending beam may be deformed upon insertion of the bone anchor relative to the implant through the through opening and may snap back into a relaxed position after having reached its final position.

The locking element may be arranged on the head of the bone anchor.

The head may comprise a proximally facing shoulder, whereby a portion with a greatest radial extension is offset towards distally from the proximal end of the bone anchor, with a guiding portion formed proximally of the shoulder.

The portion with the greatest radial extension may comprise a cantilever or beam, the cantilever or beam being the locking element.

The locking element of the system cooperates, if arranged on the bone anchor, with corresponding surface portions on the implant, for example within the through opening, and, if arranged in the through opening, it cooperates with corresponding surface portions of the anchor, for example of the anchor head. Such locking surfaces are in particular inner surfaces of grooves or depressions or portions of a distal surface of the implant or of a proximal surface of the anchor.

Especially, the locking element and the corresponding surface portion may be equipped to prevent a backing out movement independent on whether the bone anchor comprises a thread or not. To this end, in contrast for example to a system as taught in US 2015/0216573, they may be equipped to be suitable both, for an insertion of the bone anchor by an axial movement of the bone anchor relative to the implant without substantial rotation and for preventing a backing-out movement of the bone anchor into a proximal direction also without substantial rotation. Thereby, the locking mechanism may be configured for cooperating together to prevent the bone anchor from backing out independent of the structures and mechanism by which it is retained in the bone tissue. Especially, it is suitable for cannulated bone anchors anchored by bone cement pressed out from the cannulation into surrounding bone tissue, or by thermoplastic material using the approach as for example taught in WO 2011/054122—independent on whether or not the bone anchor has an additional outer thread.

The anchor may have a smooth outer surface, for example over at least 20%, especially for example over more than 50% of its length. Moreover, it may not have an outer screw thread in this area with a smooth outer surface.

The bone anchor may be a fenestrated bone anchor, wherein the shaft further comprises a longitudinal cavity extending in the direction of the axis from the proximal end towards the distal end and at least one lateral channel extending through a wall of the shaft from the axial cavity to the circumferential surface.

For guiding the movement and tensioning of the locking element on moving the bone anchor in a distal direction in the through opening, the locking element may to this end comprise a ramp leading parallel to the direction of this movement from a lower to a higher level, wherein, if the locking element is arranged on the bone anchor, the higher level is situated proximally of the lower level, and, if the locking element is arranged in the through opening, the higher level is arranged distally of the lower level. The locking element and the locking surfaces may be designed for a loose fit in the locked position.

The locking element of the system according to the second aspect may be constituted as a resilient beam being supported on one beam end (resiliently pivoting cantilever) or on two opposite beam ends (resiliently bending beam), wherein the resilient beam is cut out of the bulk of the bone anchor or the implant, i.e. it is delimited by a void extending at least along a beam length and, in the case of a sufficiently thin bulk portion, extends right through this bulk portion (beam thickness substantially the same as local bulk thickness), or, in the case of a thicker bulk portion, extends additionally underneath the beam (beam thickness constituting part of local bulk thickness).

The locking element may be such that no substantial plastic deformation or even disruption occurs. In the relaxed position, which corresponds to the locking position, the locking element may be in its initial position relative to the shaft and head, i.e., the position the locking element had before insertion in the through opening.

In a relaxed position, the resilient locking element protrudes from a general level of a surface portion of the bone anchor or the through opening in which it is situated. On moving the bone anchor into its final position relative to the through opening, the resilient locking element is pivoted or bent out of the relaxed position to a resiliently tensioned position in which it does not protrude or protrudes less from the general level of the surface of the anchor or the through opening, and, when the final position is reached, it snaps back into its relaxed position.

In a special group of embodiments, the head forms a radial protrusion in a vicinity of the proximal end of the bone anchor, for example a ring-shaped protrusion. In this special group of embodiments, the resilient locking element forms part of this radial protrusion, and the corresponding surface portions of the implant are within the through opening. Especially, the resilient locking element may form an arc-shaped beam with a radius of curvature corresponding to the radial distance from the bone anchor axis. Such beam may be supported on both sides and thereby form part of the ring-shaped protrusion in a manner symmetrical with respect to twisting directions. A locking protrusion forming the ramp and, on a proximally facing side, the locking surface, may protrude radially from the beam, for example in a middle position between the sides on which the beam is supported.

This configuration has not only been found to have a very good ratio between used material and space on the one hand and stability provided on the other hand. It is also advantageous in terms of manufacturing, since the structure may be manufactured also by material removing processes, for example by a material removing tool that cuts the arc-shaped void radially-inwardly of the arc-shaped beam into the material of the ring-shaped radial protrusion that forms the head.

In a further group of embodiments, which includes embodiments of the mentioned special group of embodiments, the head of the bone anchor may form a proximally facing shoulder, whereby the portion with the greatest radial extension—for example including the arc-shaped beam in embodiments of the special group—is offset towards distally from the proximal end of the bone anchor. Such offset may be substantial, i.e. sufficient for the bone anchor portion proximally of the shoulder having a guiding function. For example, the offset may be by at least 0.3 mm or at least 0.5 mm or at least 0.7 mm. The portion proximally of the shoulder may form a substantially cylindrical or possibly slightly conical (taper angle for example at most 10°) guiding surface.

This structure with the proximally facing shoulder firstly brings about the potential advantage that the bone anchor may be inserted by a method that mechanically loads the proximal end of the bone anchor, for example by hammering. Secondly, a hollow guiding tool, such as a guiding tube or guiding sleeve, may be put over the bone anchor portion proximally of the shoulder, with its distal end resting against the shoulder and being supported thereby. Such hollow guiding tool may be used to insert material—such as a thermoplastic element in a solid state or also bone cement—into a longitudinal cavity of the bone anchor and/or may be used to guide a tool by which energy is coupled into material inserted at least partially into such cavity. Alternatively or in addition, such guiding tool may reach into the anchor resting on the bone anchor portion proximally of the shoulder.

Therefore, the present invention also concerns a set of parts that comprises a system as described and claimed in the present text and in addition comprises a hollow guiding tool adapted to be put be put over the bone anchor portion proximally of the shoulder, with its distal end resting against the shoulder and being supported thereby.

In embodiments, the bone anchor is capable of being inserted by a movement in essentially axial direction, without substantial rotation. Thus, in these embodiments the bone anchor is free from any outer threads or similar and does not need any structures for a subjecting the bone anchor to rotation.

The implant and/or the bone anchor of the system are both made of a medically acceptable metal, polymer or ceramic material, wherein particularly the one of the bone anchor and the implant (or the part thereof comprising the through opening) which comprises the locking element may be manufactured using an additive manufacturing process (3D printing process). Bone anchor and/or implant (or part thereof), especially the one that comprises the locking element, may be made of e.g. a suitable titanium alloy, a CoCr-alloy or a molybdenum-rhenium alloy and may be manufactured using a selective laser sintering a process, a selective laser melting process or an electron beam melting process.

According to a third aspect, a load-bearing bone implant is provided, the bone implant being suitable for being implanted in a human or animal patient between surfaces of live bones or bone fragments, suitable for bearing at least temporarily loads acting between these bones or bone fragments, and suitable for being integrated between the bones or bone fragments by bone growth after surgery.

In embodiments, the bone implant according to the third aspect belongs to a system that also comprises a bone anchor according to the first aspect, and/or belongs to a system according to the second aspect.

The bone implant may be a load-bearing bone implant suitable for being implanted in a human or animal patient between surfaces of live bones or bone fragments, suitable for bearing at least temporarily loads acting between these bones or bone fragments, and suitable for being integrated between the bones or bone fragments by bone growth after surgery. Such bone implants are applicable in surgical procedures such as e.g. spinal fusion, arthrodesis and osteotomy.

The bone implant may comprise a porous implant body with an open porosity constituting throughout the porous implant body a three-dimensional network of porosity channels of dimensions suitable for bone ingrowth, which porous implant body constitutes two substantially opposite body surfaces (ingrowth surfaces) to be positioned against the live bone tissue on implantation. In addition to the porosity channels, the porous implant body comprises a plurality of supply channels, wherein each one of the supply channels has a mouth in at least one of the ingrowth surfaces and extends into or for example through the porous implant body, for example about parallel to the force lines of the force field acting on the implant in the implanted state. Therein the supply channels have, at least over part of their length, cross sections larger than the cross sections of the porosity channels, but small enough for being bridgeable by spontaneous bone growth without bone growth enhancing material positioned in the channels.

The supply channels may extend through the porous implant body between the opposite ingrowth surfaces. They may extend substantially parallel to said forces. In addition or as an alternative, they may extend parallel to each other.

The porosity channels of the porous implant body of the implant according to the third aspect have cross sections with diameters in the range of 0.1 to 0.7 mm diameter, for example in the range of 0.4 to 0.6 mm. The supply channels have, at least over a part of their length, cross sections of any suitable form of which the smallest dimension is in the range of 1 to 3 mm or in the range of 1.2 to 3 mm. A mean supply channel diameter (i.e. the diameter of a circle having the same area as the cross sectional area of the supply channel if the cross section is constant; if it is not constant the definition refers to the such determined diameter averaged over the supply channel's length) is between 1.5 mm and 3 mm, especially between 1.7 mm and 2.5 mm. In many embodiments, the cross section is constant along the supply channel's length, with the possible exception of an enlarged mouth portion (see below), in which case the above definition of the mean diameter applies to the channel portions excluding the mouth portion.

Throughout the porous implant, the supply channels may have distances from each other and for example from the peripheral surface portions in the range of 2 to 6 mm, for example in the range of 3 to 5 mm. The total volume of the porosity channels and the supply channels together constitute 40-80% of the volume of the porous implant body, for example 60-80%).

Especially, the supply channels may be arranged to be capable of forming an arrangement of equidistant of supply of the porosity channels in that the largest distance (measured between their respective surfaces) between neighboring supply channels may, with the possible exception of a central region having a cavity of the above-mentioned kind, correspond to at most 6 mm or at most 5 mm or corresponds to at most 3.5 times or at most 3 times or at most 2.5 times their mean diameter (the ratios being a possible design criterion for all different kinds of implants described in this text and especially being a good measure for smaller implants, for example for cervical applications). In addition or as an alternative, a maximum distance between peripheral supply channels and the lateral surface may correspond to at most 3.5 times or at most 3 times or at most 2.5 times their mean diameter, whereby the arrangement of the channels follows the outer contour of the implant.

Especially, the supply channels, for example all of them, may have the same distances from each other and may be arranged in a regular pattern, for example in a hexagonal pattern.

The design of the porous implant body of the bone implant according to the third aspect is based on the following findings. Bone ingrowth into porous implant body structures more or less mimicking the trabecular structure of natural bone tissue is successful only to a depth of 1 to about 3 mm (probably limited by the supply of the ingrown bone tissue by diffusion only). Spontaneous bone growth, i.e. bone growth within weeks after surgery, will bridge gaps of a width of not more than about 3 mm. In larger cavities (all dimensions larger than about 3 mm), bone growth will primarily cover the walls of the cavity, wherein in such cavities having a smallest dimension of not more than about 10 mm (7 to 10 mm), long term bone growth, i.e. bone growth within months, will be able to bridge the cavity, and wherein even larger cavities (all dimensions larger than 7 to 9 mm, critical size defect) do not fill with bone tissue at all, unless they are filled with a bone growth enhancing material such as e.g. bone graft material. No or hardly any ingrowth of bone tissue into the implant is found from implant surfaces other than the implant surfaces (ingrowth surfaces) being, in the implanted state, in direct contact with live bone tissue of the patient, i.e. ingrowth of bone tissue into the implant is highly anisotropic.

The dimensions of the supply channels are chosen in view of such findings about physiological relations. It has been found that especially, but not only, for implants having a thickness (extension perpendicular to the ingrowth surfaces) of more than 10 mm, especially around 20 mm—which is a characteristic dimension for spinal fusion implants especially for the lumbar and thoracic vertebrae—the vasculature and lymph structures will not grow sufficiently deep into the supply channels if their diameter is about 1 mm or lower. Thus, physiological relations yield a lower limit of about 1.2 mm, 1.5 mm, in examples even 1.8 mm for the supply channel diameter. An upper limit of typically 3 mm or 2.5 mm is set both, by mechanical/geometrical considerations (if the supply channels are too wide, there is not sufficient space for there also being load bearing structures and a sufficient number of supply channels as well as sufficient volume for the porosity channels) and by the above-explained findings on spontaneous bone growth. For smaller implants, for example for cervical applications, having a lateral width of for example not much more than 10 mm and having a height of only a few millimeters (for example just 4 mm), a diameter of the supply channels may be lower than 2 mm and for example in the range between 1 mm or 1.2 mm and 2 mm, and the above-mentioned considerations for the ratio between distances and channel diameters may apply.

Based on the above listed findings, therefore, the supply channels of the porous implant body of the implant originate from the ingrowth surfaces, have cross sections large enough for allowing ingrowth of bone tissue including supply means such as in particular vasculature and lymph channels and small enough for being bridged, i.e. completely filled with bone tissue, and, if suitably distanced from each other (and possibly from ingrowth surfaces) throughout the porous implant body enable bone growth in all locations thereof. Furthermore, it is found that supply channels longer than about 20 mm are for example equipped with an enlarged mouth portion.

An aspect ratio of the supply channels (i.e. a ratio between their length across the implant and their mean diameter) may be chosen to be not higher than about 15 or not higher than about 10 or about 8. In embodiments with the enlarged mouth portion, the length of the supply channel for determining the aspect ratio in this is measured excluding the enlarged mouth portion. For larger aspect ratios than these values, ingrowth into the supply channels has been found to be sometimes incomplete.

The orientation of the supply channels along the force lines is advantageous from a mechanical point of view. In most considered cases, these force lines extend substantially parallel to each other from one of the ingrowth surfaces to the opposite other one. Therefore, in a possible arrangement, the supply channels extend substantially parallel to each other from one ingrowth surface towards, for example reaching all the way to, the other one, where the above-discussed distances between the supply channels may apply. In embodiments, for being able to supply the largest porous volume with the smallest number of supply channels, the latter may be arranged in an approximately hexagonal system, wherein, in a cross section through the channel arrangement, each channel has six neighboring channels at an approximately same distance of for example 5-6 mm.

In addition or as an alternative to being arranged in an approximately hexagonal system, the channels may be arranged in a pattern that follows the outer contour and, if the implant comprises a cavity, a contour defined by the limits of the cavity. The pattern may therefore comprise a (first) row of outer, peripheral supply channels following the outer contour and at least one further, second row of supply channels parallel to the first row, the position of the supply channels of the second row for example being staggered with respect to the positions of the supply channels of the first row to yield an approximately hexagonal system. The implant may comprise further (third, for example fourth or even more) rows of supply channels successively increasing distances from the peripheral surface portions.

The implant may comprise, in addition to the porous implant body, a support frame which partially surrounds and possibly also penetrates the porous implant body. The support frame is made of a suitable, for example non-porous material. The support frame may further comprise at least one of a member constituting at least part of a proximal face of the implant, a member extending along an edge around one of the ingrowth surfaces, a member extending in one of the ingrowth surfaces, and a member extending through the porous implant body.

The support frame may further comprise surface elements consisting of a substantially non-porous material, being arranged in at least one of the ingrowth surfaces of the porous implant body and flush with the latter. Such surface elements may surround mouths of selected ones or all of the supply channels

The support frame may constitute at least part of a proximal implant surface (trailing surface on implantation, usually not an ingrowth surface) and members e.g. running along edge regions of the porous implant body, which edge members e.g. surround ingrowth surfaces without protruding from the latter, extend in ingrowth surfaces, or penetrate the porous implant body. Outer surfaces of the support frame may be equipped with a per se known osseointegration enhancing surface structure or coating, at least where they are to be positioned against surfaces of live bone tissue and where osseointegration is desired.

The bone implant may comprise further elements of a substantially non-porous material. Such further elements are e.g. surface elements, which are arranged in the ingrowth surfaces and flush therewith and may serve for facilitating implantation by reducing friction between the ingrowth surface and the surface of the live bone tissue without impairing the direct contact between the porous implant body and the live bone tissue. The surface elements e.g. surround mouths of supply channels and therewith help to prevent mechanical damage of the mouth edges. Further such elements may also be struts extending along the wall of selected ones of the supply channels and serving as stiffening and supporting means.

While the dimensions of the members of the support frame depend on the type and size of the implant and on the load the latter has to bear when implanted, the surface elements have dimensions which are as small as possible. In a medium interbody fusion implant, the members of the support frame have e.g. widths and depth (into the porous implant body) in the range of a few millimeters (2 to 5 mm) and the surface elements have widths and depths of about one millimeter (0.8 to 1.5 mm). The same applies to the struts.

The implant may, in the same manner as known such implants, further comprise a larger cavity to be filled with bone growth enhancing material, wherein this cavity may, in a per se known manner, reach from one ingrowth surface to the other one. Furthermore, the implant may comprise per se known mechanical retention means.

The porous implant body is made of a suitable, medically approved metal, polymer or ceramic material, e.g. titanium (grade 1-4), titanium alloy (e.g. Ti-6Al-4V or Ti-7Al-11Nb), resorbable magnesium alloys, PEEK, polylactide (resorbable), biocomposites, zirconium oxide, aluminum oxide or mixtures of the two oxides, or calcium phosphate (tri-calciumphosphate, hydroxyapatite, both resorbable). The porous implant body may be manufactured using an additive manufacturing method, e.g. selective laser sintering, e-beam melting, or fused deposition (in particular for polymer and ceramic material). For strengthening the porous structures manufactured e.g. with the named 3D-printing methods they may be compacted by HIP-processes. For rendering them more bio-active, they may be surface treated in galvanic processes (e.g. etching) or non-galvanic processes (e.g. nano-deposition).

The support frame and possibly also further elements and retention means of the implant may be made of the same material as the porous implant body and may be manufactured in the same additive process as the porous implant body such forming one integral part with the latter.

Selected ones of the supply channels may comprise an enlarged mouth portion, wherein the enlarged mouth portion may have dimensions in the range of 3 to 10 mm.

The implant according to the third aspect is particularly suitable for applications in which the ratio between implant surface and implant volume is relatively small (relatively large implant bulk), in particular it is suitable for applications requiring an implant thickness (distance between opposite ingrowth surfaces) in the range of 4 to about 30 mm. Furthermore, it is particularly suitable for applications in which provision of permanent mechanical retention structures is limited, and for which long-term success of the corresponding surgical procedure is highly dependent on successful bone growth after surgery. This is e.g. the case for interbody fusion devices used in spine surgery, for various implants used in other arthrodesis methods, and for wedge-shaped implants used in various osteotomy procedures.

Thus, the implant may constitute an intervertebral fusion implant, an osteotomy wedge, or an implant suitable for an arthrodesis procedure.

If the implant constitutes an intervertebral fusion implant, the implant, a first ingrowth surface of the ingrowth surfaces may be a cranial ingrowth surface and a second ingrowth surface of the ingrowth surfaces may be a caudal ingrowth surface, the implant comprising a plurality of lobes, each comprising a through opening for receiving a bone anchor.

In this. the lobes may be arranged protruding from an anterior and face of the implant, wherein a first and a second lobe of the plurality of lobe protrudes cranially above the cranial ingrowth surface, and wherein a third and a fourth lobe of the plurality of lobes protrude caudally below the caudal ingrowth surface.

One of a plurality of embodiments of the bone implant is an interbody fusion implant, which may be of the stand-alone type or of the type which is combined with further instrumentation such as e.g. systems of pedicle screws and spinal rods. Known such implants are e.g. disclosed in the publication WO2010/096942, which is enclosed herein in its entirety by reference. The devices as disclosed in the named publication comprise an interbody implant and, if of the stand-alone type, a plate which constitutes a separate element or is fixedly arranged on the interbody implant. The plate is fixated on the anterior or on a lateral side of the two vertebral bodies between which the interbody implant is positioned. For the fixation of the plate, in particular fenestrated headed bone anchors are proposed, which bone anchors are driven into the bone tissue of the vertebral body through openings provided in the plate and are anchored in the vertebral body e.g. with the aid of a material having thermoplastic properties and being liquefied by application of e.g. vibration energy within a longitudinal cavity of the anchor and pressed to the outside of the anchor through lateral channels (fenestration) to re-solidify for example within the trabecular structure of the bone tissue surrounding the anchor or between the bone tissue and the anchor.

The invention also concerns an intervertebral fusion device comprising a bone implant according to the third aspect and at least one bone anchor, wherein the bone implant comprises, in addition to the porous implant body and the support frame, a bone plate with at least one through opening for receiving the at least one bone anchor.

In such an intervertebral fusion device at least the porous implant body, the support frame and the bone plate may consist of the same material and may be manufactured from a single piece of the material or by additive manufacturing.

In embodiments, the intervertebral fusion device belongs to a system according to the second aspect.

### BRIEF DESCRIPTION OF THE DRAWINGS

The invention is described in further detail in connection with the appended FIGS., wherein:

FIGS. 1A/B/C illustrate an exemplary embodiment of the fenestrated bone anchor, the embodiment comprising three lateral channels with pear-shaped cross sections;

FIG. 2 is a plan view of the fenestrated bone anchor according to FIGS. 1A/B/C (viewing direction towards the head of the anchor)

FIGS. 3 and 4 are cross sections through further exemplary embodiments of the bone anchor, the embodiments comprising an increase in wall thickness in a direction towards the lateral channels;

FIGS. 5A/B/C illustrate the principle of an exemplary locking element of the system, the illustrated locking element having the form of a resilient cantilever;

FIGS. 6A/B/C illustrate the principle of an exemplary locking element of the system, the illustrated locking element having the form of a resilient bending beam;

FIGS. 7A/B/C shows an exemplary embodiment of a bone anchor of the system, the bone anchor comprising a locking element in the form of a resilient cantilever, the cantilever length extending substantially parallel to the movement of the bone anchor relative to the through opening;

FIGS. 8A/B show an exemplary embodiment of a bone anchor of the system, the bone anchor comprising a locking element in the form of a resilient cantilever, the cantilever length extending substantially perpendicular to the movement of the bone anchor relative to the through opening;

FIGS. 9A/B/C show an exemplary embodiment of a bone anchor of the system, the bone anchor comprising a locking element in the form of a resilient bending beam, the beam length extending substantially perpendicular to the movement of the bone anchor relative to the through opening;

FIGS. 10 and 11 illustrate the principle of a bone implant, the bone implant being shown in section perpendicular to ingrowth surfaces of the porous implant body;

FIGS. 12, 13, 14 are cross sections of exemplary embodiments of supply channel arrangements;

FIG. 15 illustrates a further exemplary embodiment of the bone implant;

FIG. 16 shows an interbody fusion device of the stand-alone type comprising a plate with through openings suitable for being fixated to vertebral bodies with the ais of bone anchors.

### DESCRIPTION OF THE PREFERRED EMBODIMENTS

In the whole of the present text the term “proximal” is used to designate a position nearer a surgeon and the term “distal” a position nearer the patient. Similarly, a proximal direction means a direction against the surgeon and a distal direction a direction against the patient or further into the patient. Each item has a proximal end and a distal end, the distal end being the leading end on implantation and the proximal end being the trailing end on implantation. Most described items have a proximal end and a distal end and an axis extending therebetween wherein this axis on implantation coincides or is parallel to the implantation direction and wherein the length of this axis may or may not be the longest extension of the item.

In the appended figures, similar elements or elements with same functions are designated with same reference numerals.

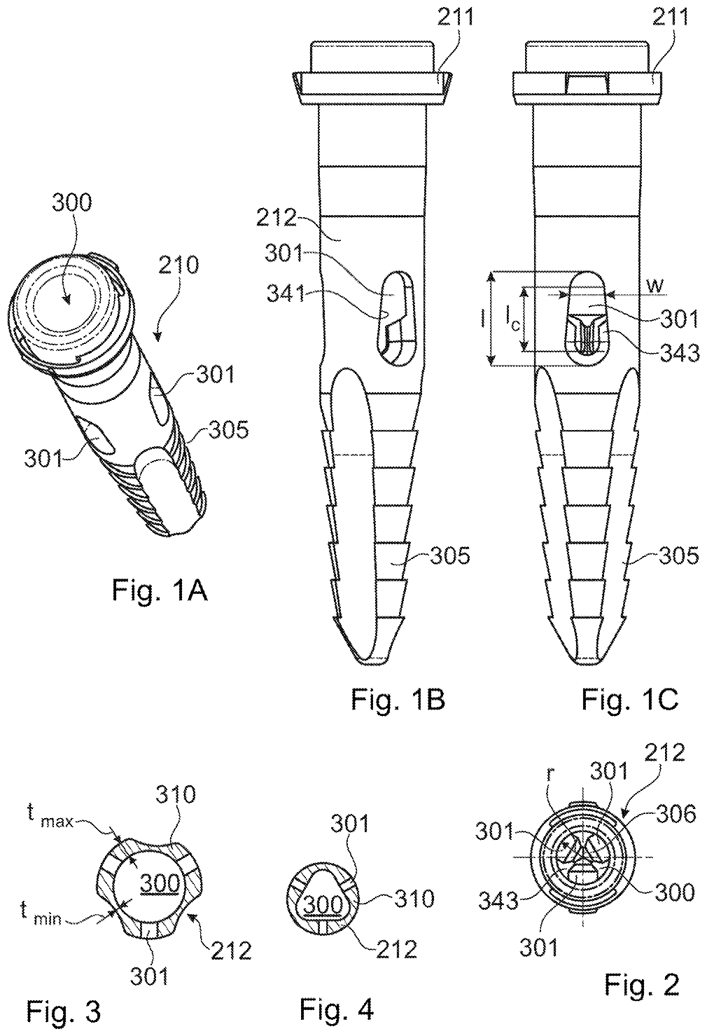

FIGS. 1A/B/C illustrate an exemplary embodiment of the fenestrated bone anchor, wherein FIG. 1A is a three-dimensional illustration of the anchor, and FIGS. 1A and 1B are lateral views differing from each other by an angle of 90° between the corresponding viewing directions. The bone anchor 210 comprises a head 211 and a shaft 212. The longitudinal cavity 300 reaches through the head 211 into the shaft to a closed distal end and it is connected with the circumferential surface of the shaft by three lateral channels 301. As described further above, the lateral channels 301 have a pear-shaped cross section, i.e. an axial length 1 (see FIG. 1C) greater than a circumferential width w. The width w decreases in a proximal direction. The cross section has in a per se known manner no sharp corners such preventing load concentrations. The lateral channels 301 have outer mouths positioned all at the same axial position situated in the proximal half of the shaft axis, the closed distal end of the longitudinal cavity 300 is situated about half way between the proximal shaft end and the distal shaft end. The circumferential surface of the distal part of the shaft is equipped with circumferential ribs constituting exemplary means for retaining the anchor in a bone opening provided for its implantation.

The overall pear shape of the lateral channels is such that the sidewalls 341 and the edges of their mouth are not parallel to each other also in a middle region (between the dotted lines in FIG. 1C), the middle region having a substantial axial extension l_{c} compared to the axial extension l of the axial channel. Also, the average circumferential width w of a proximal half of the lateral channel (above the dashed line in FIG. 1C) is substantially smaller than the average circumferential width of the distal half. An angle between the sidewalls and the longitudinal axis in the depicted embodiment is about 5°

FIG. 2 is a plan view of the fenestrated bone anchor as shown in FIGS. 1A/B/C (viewing direction from the head towards the tip of the anchor). It illustrates the distal end of the longitudinal cavity, which is equipped with relatively sharp edges 306 or peaks serving as energy concentrators during the process of liquefaction of a material having thermoplastic properties being positioned in the longitudinal cavity and being pressed against the distal end of the longitudinal cavity, while ultrasonic vibration energy is applied to it. Also visible in FIG. 2 are the inner mouths of the lateral channels 301. Further exemplary embodiments of distal cavity ends suitable for the fenestrated bone anchor are e.g. disclosed in the publication WO2011/054122, the disclosure of which is enclosed herein in its entirety by reference.

More in concrete, the closed distal end of the longitudinal cavity 300 has a shape different from circularly symmetrical about the axis. Rather, the channels 301 have an inner portion at radial positions within a radius of the longitudinal cavity 300 in addition to an outer portion formed by the (pear-shaped) opening (exit opening) in the wall around the cavity 300. Thus, the distal end of the longitudinal cavity is angularly structured so as to direct different portions of the material (material having thermoplastic properties or bone cement) to the different exit openings.

In the embodiment shown in FIGS. 1A-2, the structures of the distal end comprise directing walls 343 that extend radially between the inner portions of the channels 301. Proximal edges of such walls may optionally in addition to serving as directing structures serve as energy directors in the above-mentioned sense.

FIGS. 3 and 4 are cross sections through the shaft 212 of further exemplary embodiments of the fenestrated bone anchor. The section plane of these cross sections is situated in the axial position of the lateral channels 301 and illustrate in particular the wall 310 between the longitudinal cavity 300 and the circumferential shaft surface. This wall has a wall thickness which increases in a circumferential direction towards each lateral channel 301 from a minimum wall thickness tₘᵢₙ in locations between the lateral channels 301 and a maximum wall thickness tₘₐₓ where the wall meets the lateral channel 301. The increase of the wall thickness is e.g. in the range of 20%. This design measure renders the cross section of the anchor shaft 212 and the cross section of the longitudinal cavity 300 to be different, wherein in the illustrated case showing three lateral channels 301 the one cross section is circular and the other one is three-lobed having a circular envelope. Accordingly, for an anchor having two lateral channels, the lobed cross section is like an oval (two-lobed), for four channels it is four-lobed, and so on.

The embodiment of wall 310 according to FIG. 3 for which the cross section of the shaft is lobed and the cross section of the longitudinal cavity is circular is advantageous when using the corresponding anchor for the above briefly described anchoring process with the aid of an element of a material having thermoplastic properties, as this element may be a simple pin having a circular cross section. On the other hand, it either necessitates a bone opening provided for the anchor having the trilobal cross section of the anchor shaft or a larger opening having a circular cross section substantially corresponding to the circular envelope or the trilobal shaft cross section. In an axial direction, in particular in a proximal direction, away from the lateral channels, the lobed cross section may change gradually into a circular cross section such guaranteeing good guidance of the anchor in an also circular through opening of e.g. a bone plate. The embodiment of the wall 310 according to FIG. 4 is advantageous for an anchor of an overall circular cross section, e.g. a fenestrated screw, but for the above-mentioned anchoring process it necessitates an element of the material having thermoplastic properties in the form a pin with a non-circular cross section.

Further embodiments of the fenestrated bone anchor differ from the embodiments illustrated in the appended figures by not comprising a head or a head of a different form, by comprising instead of three lateral channels only one or e.g. 2 or 4 lateral channels, by the lateral channels not being situated further proximal but in the middle of the axial shaft length or further distal, by the longitudinal cavity reaching right through the shaft and having an open distal end, by not having a general circular shaft cross section but a shaft cross sections as e.g. listed in the introductory part of the present disclosure, by the lateral channels being situated not in a same axial position but in differing axial positions, and/or by comprising different or no retention means as e.g. listed in the introductory part of the present disclosure. All the named alternative features can be selectively used or combined for a plurality of further exemplary embodiments of the fenestrated bone anchor and designed for specific applications.

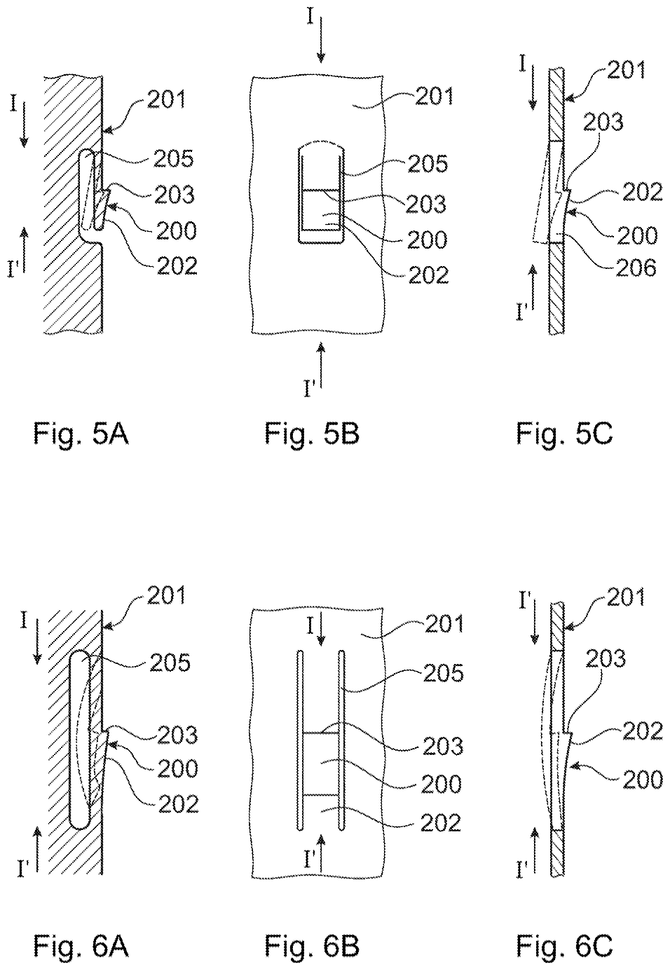

FIGS. 5A/B/C and 6A/B/C illustrate each in a schematic manner the principle of exemplary embodiments of locking elements 200 suitable for a any embodiment of the fenestrated bone anchor, wherein the bone anchor cooperates with a through opening provided in an implant, bone anchor and implant forming together a system. Each one of these FIGS. shows a surface portion 201 of a bone anchor or through opening of an implant belonging to the system, wherein the locking element 200 is arranged within this surface portion 201. FIGS. 5A, 5C, 6A and 6C are sections through the surface portion 201 and the locking element 200, the section plane being oriented substantially parallel to the axis of the bone anchor or the through opening, or to the implantation direction respectively. These FIGS. show the locking element 200 in its relaxed position (uninterrupted lines), in which it protrudes from the general level of the surface portion 201, and in its resiliently tensioned position (interrupted lines) in which it protrudes less or not at all from the general level of the surface portion 201 (flush or countersunk). FIGS. 5B and 6B are plan views of surface portion 201 and locking element 200. The surface portions 201 may be portions of the circumferential surface of either the bone anchor head or the bone anchor shaft and are substantially convex. However, the illustrated surface portions 201 may also be portions of the inside surface of the through opening of the implant and, in such a case, are substantially concave. Locking elements arranged on the bone anchor (shaft or head) are moved relative to the through opening in a direction illustrated with an arrow I (implantation direction). Locking elements arranged in the through opening are moved relative to the bone anchor in an opposite direction indicated with the arrow I′.

The locking element 200 comprises a protrusion with a guiding ramp 202 and a locking surface 203, wherein the guiding ramp 202 is situated upstream or downstream of the locking surface, depending on the moving direction I or I′. The illustrated locking elements comprise protrusions with ramps 202 and locking surfaces 203 extending over the full width of the locking element. Alternatively, the protrusion may be narrower.

The locking elements illustrated in FIGS. 5A/B/C have the form of a resilient cantilever pivoting in a plane substantially parallel to the directions I and I′ or to the axis of the bone anchor or the through opening respectively. The void 205 delimiting the cantilever extends along its length and on one side along its width also, and, depending on the thickness of the bulk beneath the surface portion 201 and on the bulk material, extends furthermore underneath the locking element (FIG. 5A) or fully through the bulk underneath the locking element (FIG. 5C).

The locking elements illustrated in FIGS. 6A/B/C have the form of a resilient bending beam with a bending movement in a plane substantially parallel to the directions I and I′ or to the axis of the bone anchor or the through opening respectively. The void 205 delimiting the bending beam extends along its length limiting its width, and, depending on the thickness of the bulk beneath the surface portion 201 and on the bulk material, extends furthermore underneath the locking element (FIG. 6C) or fully through the bulk underneath the locking element (FIG. 6C).

Dimensions of the cantilever or bending beam of the locking elements need to be adapted to the bulk material and bulk thickness in the region in which the locking element is situated. Generally speaking, deformation of the cantilever or bending beam needs to remain in the elastic range (less than 1%). For locking elements as shown in FIGS. 5A and 6A, there is more design freedom regarding thickness and therewith length of the locking element than there is for the locking elements as shown in FIGS. 5C and 6C, for which the thickness of the locking element is given by the bulk thickness. Whereas the embodiments of FIGS. 5A and 6A only require a minimum bulk thickness, the embodiments according to FIGS. 5C and 6C can only be realized within a limited range of bulk thickness (substantially limited to cannulated bone anchor). Applicability of the embodiments according to FIGS. 5C and 6C is further limited by the fact that these locking elements constitute through openings through e.g. a wall of a cannulated bone anchor which, in the location of the locking element may not be tolerable, in particular when the anchor is used e.g. for an augmentation process with the aid of a flowable material such as a bone cement.

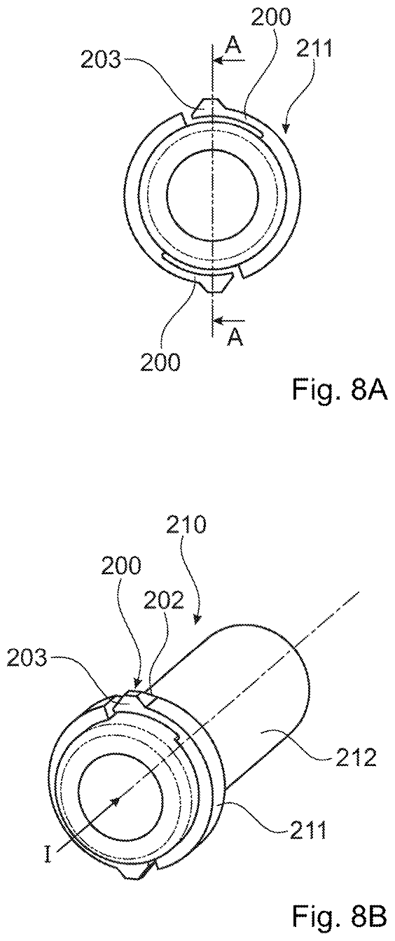

The locking elements as illustrated in FIGS. 5A/B/C and 6A/B/C comprise cantilevers or bending beams with a length extending substantially parallel to the directions I and I′ or to the axis of the bone anchor or the through opening on which they are arranged. This is not a necessary feature of the locking element of the system. The lengths of such a cantilever or bending beam can also extend at an angle to the directions I and I′ or substantially perpendicular to them, wherein ramp and locking surface still have to be arranged following each other in the direction of the movement of bone anchor and through opening relative to each other. This means for a cantilever or bending beam length extending substantially perpendicular to the direction of this movement, that ramp and locking surface are arranged beside each other over the width of the cantilever or bending beam width. Two exemplary embodiments of locking elements in the forms of cantilever and bending beam having a length extending substantially perpendicular to the named moving direction or anchor axis respectively are illustrated in FIGS. 8A/B and 9A/B/C.

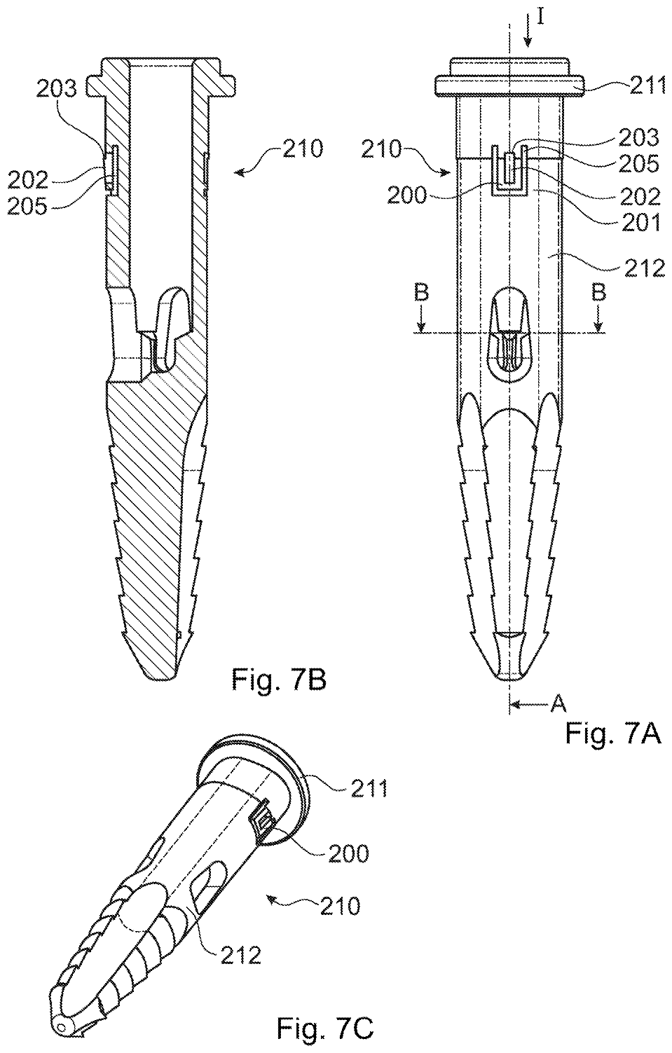

FIGS. 7A/B/C illustrate an exemplary embodiment of a bone anchor of the system. The bone anchor is shown viewed from a lateral side (FIG. 7A), sectioned along its axis (FIG. 7B), and in a three-dimensional representation (FIG. 7C). The bone anchor 210 comprises a head 211 and a shaft 212 on which two opposite integrated locking elements 200 of the type being illustrated in FIGS. 5A and 5B are provided. Each one of the locking elements 200 has the form of a resilient cantilever surrounded on three sides by a void 205 and having a length extending parallel to the anchor axis and the direction I. The locking element further comprises a ramp 202 and a locking surface 203 which, as best seen from FIG. 7A, have a width smaller than the width of the cantilever, and which, as seen best from FIG. 7B, are arranged adjacent to each over the cantilever length, wherein, in the direction I, the ramp 202 is arranged downstream of the locking surface 203.

FIGS. 8A/B show a further exemplary embodiment of the bone anchor 210 of the system. The bone anchor 210 is shown in a three-dimensional representation (FIG. 8A) and in a plan view viewed against its proximal surface (FIG. 8B). The bone anchor 210 comprises a head 211, a shaft 212 (only partially shown in FIG. 8B), and arranged on the head, two locking elements 200 in the form of cantilevers as illustrated and described in connection with FIGS. 8A and 8B. Other than shown in FIGS. 8A and 8B, the cantilever length is not oriented parallel to the direction I or the anchor axis respectively, but it is oriented perpendicularly to the latter, i.e. the cantilever length extends circumferentially and the ramp 202 and the locking surface 203 are arranged adjacent to each other over the cantilever width, with the ramp 202, relative to the direction I being arranged downstream of the locking surface 203.

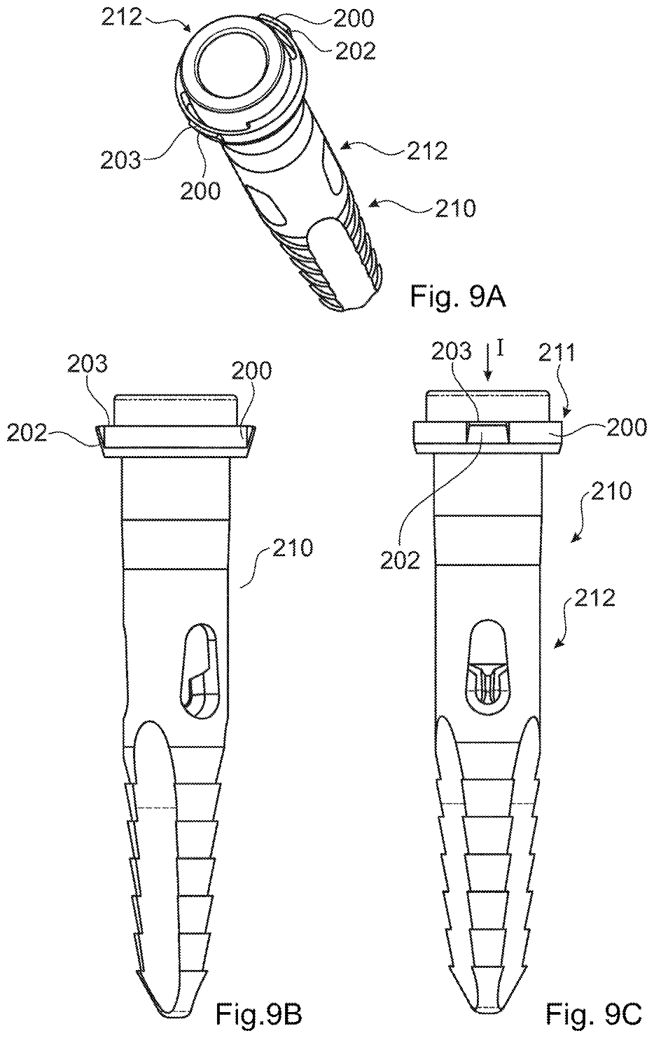

FIGS. 9A/B/C show a further exemplary embodiment of the bone anchor 210 of the system. The bone anchor is shown in a three-dimensional representation (FIG. 9A) and in two lateral views (FIGS. 9B and 9C, angle between the two viewing directions of) 90°. The bone anchor 210 comprises again a head 211 and a proximally cannulated shaft 211 and, arranged on the head 211, two opposite locking elements 200 in the form of bending beams as illustrated in FIGS. 6A and 6B. Other than in FIGS. 6A and 6B, the beam length does not extend substantially parallel to the anchor axis but substantially perpendicular to it, i.e. circumferentially. extending substantially perpendicular to the anchor axis or the direction I respectively. Therefore, the ramp 202 and the locking face 203 of the locking elements 200 are arranged adjacent to each other over the beam width as above further described in connection with FIGS. 8A/B. In all previous FIGS., the bone anchor and therewith also the through opening of the implant have substantially circular cross sections. Of course, these cross sections may have other forms, such as e.g. oval, rectangular, polygonal with sharp or blunt edges. This means that the surface portions in which the locking elements are provided are not necessarily curved surfaces.

All bone anchors shown in FIGS. 7 to 9 are fenestrated bone anchors comprising a longitudinal cavity and lateral channels and are suitable for being retained in the bone tissue by being augmented using a bone cement or a material having thermoplastic properties. This is not a necessary feature of the system. The bone anchor of this system may comprise any per se known retention means, i.e. the bone anchor can be a bone screw (solid, cannulated or fenestrated) or it can comprise retention means in the form of ribs, edges, resilient elements etc. The bone anchor of the system may also be a simple headed pin with a possibly rough circumferential surface and being suitable to be retained in the bone tissue by a press fit.

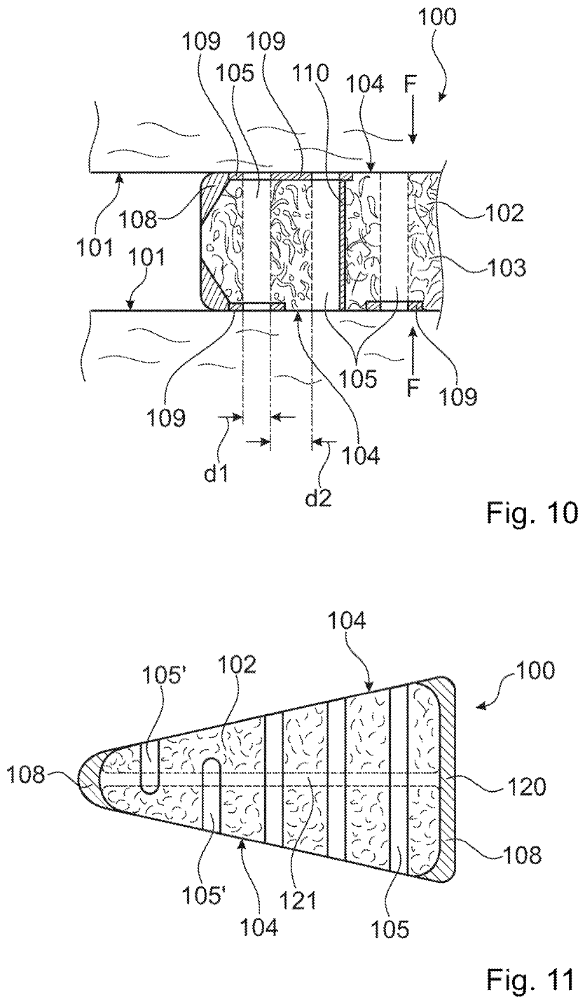

FIGS. 10 and 11 illustrate the principle of the structure of the porous implant body of the bone implant, wherein FIG. 10 illustrates an implant example with two opposite substantially parallel ingrowth surfaces, e.g. an interbody fusion implant, and FIG. 11 illustrates an implant example with two ingrowth surfaces extending at an angle relative to each other, e.g. a wedge-shaped implant as often used in osteotomy procedures.

The implant 100 shown in FIG. 10 in section perpendicular to the live bone surfaces 101 between which it is implanted is e.g. an interbody fusion implant, the live bone surfaces 101, in such a case, being suitably prepared lower and upper surfaces of neighboring vertebral bodies, wherein forces acting on the implanted implant are mainly compressing forces (arrows F) acting substantially perpendicular to the live bone surfaces 101 or the ingrowth surfaces 104 respectively. The implant 100 comprises a porous implant body 102 of an open porosity constituting a three-dimensional network of porosity channels 103. Ingrowth surfaces 104 are opposite surfaces of the porous implant body 102 which, in the implanted state of the implant, are in direct contact with the live bone surfaces 101. The implant body further comprises a plurality of supply channels 105 extending between the two ingrowth surfaces 104 substantially in the direction of the forces acting on the implanted implant. Whereas the porosity channels 103 have cross sections with diameters in the range of a few tenths of a millimeter, the supply channels 105 have cross sections of a diameter d1 of a few millimeters (such as 1-3 mm). The distances d2 between the supply channels 105 are in the range of 2 to 6 mm (for example 3 to 5 mm). For example, but not necessarily, the distances d2 between the supply channels 105 are, throughout the porous implant body, about constant, and, in no case, are larger than about 5 to 6 mm. This means that the supply channels 105 may extend through the porous implant body 100 in a substantially regular pattern.

Also shown in FIG. 10 are substantially non-porous edge members 108 of a support frame surrounding at least partially the porous implant body 102, as well as substantially non-porous surface elements 109 arranged to extend flush in the ingrowth surfaces 104 and e.g. surrounding mouths of supply channels 105 and/or extending between such mouths. Also shown in FIG. 11 is a strut 110 extending along the wall of one of the supply channels 105, wherein more than one strut 110 may be provided in one and the same supply channel 105 and wherein distances between struts are to be in the range of 0.5 to 2 mm. Surface elements 109 and struts 110 may be provided for each one of the supply channels 105 or for selected ones only.

FIG. 11 is a very schematic representation (again in a section perpendicular to the ingrowth surfaces 104) of an exemplary wedge-shaped implant 100, which implant is possibly suitable for use in an osteotomy operation. The main features of the implant are the same as the features of the implant as shown in FIG. 10, namely the porous implant body 102 with ingrowth surfaces 104 (in FIG. 11 two ingrowth surfaces at an angle relative to each other), supply channels 105 extending from one ingrowth surface 104 towards the other one, and members 108 of a support frame. In addition to the supply channels 105 extending from one ingrowth surface 104 to the other one and having a mouth in either one of the ingrowth surfaces 104, there are illustrated two blind supply channels 105′, which only have one mouth and a closed end opposite the mouth. For guaranteeing satisfactory supply for bone ingrowth beyond the closed end, the dead end is to be positioned not more than 5 to 6 mm (for example between 3 to 5 mm) distanced from the neighboring ingrowth surface (same as distance between supply channels). The support frame of the implant according to FIG. 11 differs from the support frame of the implant according to FIG. 10 in that it comprises, in addition to an edge member 108 arranged at the distal end of the porous implant body 102, a member 120 constituting a proximal implant surface and at least one central member 121 extending through the implant body. The implant according to FIG. 11 may or may not comprise surface elements or struts (none shown) as described further above in connection with FIG. 10.

Any embodiment of the bone implant may comprise in addition or alternatively to supply channels extending from one ingrowth surface to another one (as shown in FIG. 10), blind supply channels as illustrated in FIG. 11.

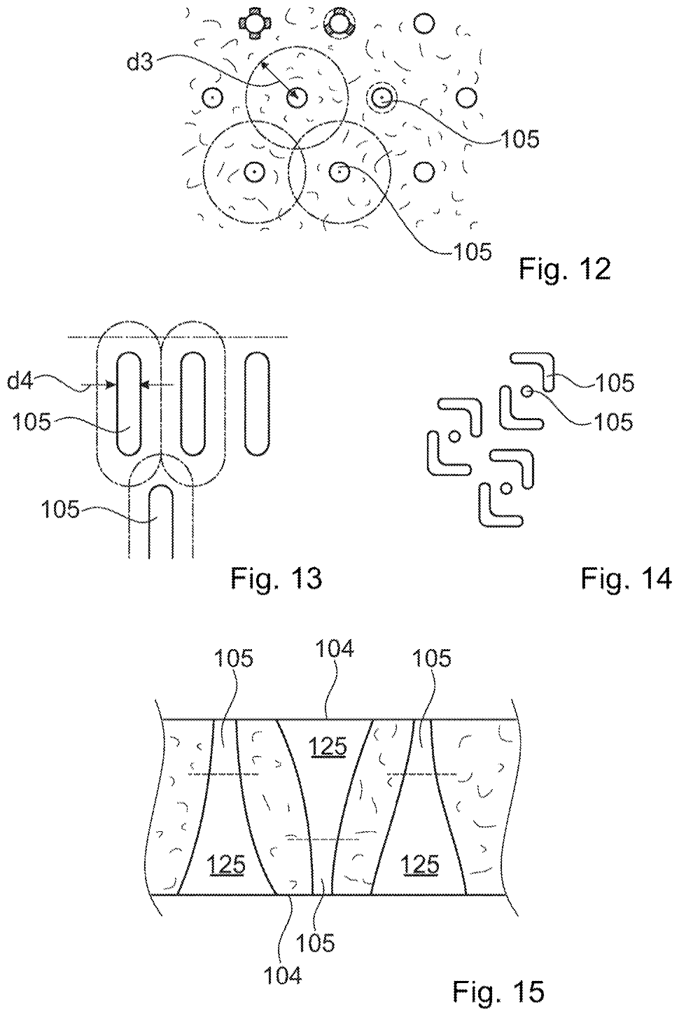

FIG. 12 further illustrates a preferred embodiment of the arrangement of supply channels 105 in a bone implant, namely the above-mentioned hexagonal arrangement. The arrangement is shown in section substantially perpendicular to the supply channels. In this arrangement, every supply channel 105 has six nearest neighbor channels, wherein all positions between the channels can be safely supplied if the distance d3 (radius of circles marked with dash-dotted lines) corresponds to the given limit of 1 to 3 mm. With the hexagonal arrangement the largest volume of porous structure can be supplied with the smallest number of supply channels.

Also shown in FIG. 12 are exemplary supply channels bring equipped with surface elements 109 surrounding supply channel mouths or connecting them and supply channels comprising varying numbers of struts 110, wherein supply channels 105 in any supply channel arrangement and of any bone implant as above described may or may not be correspondingly equipped.

In all previous FIGS. the supply channels have a substantially circular cross section. This is not an obligatory feature of the bone implant. On the contrary, in all embodiments of the bone implant, the supply channels may have cross sections of any other form, as long as for these cross sections the smallest dimension is in the range of 1 to 3 mm. This means that the supply channels may have e.g. square, rectangular, slot-shaped, triangular, oval, lobed, pentagonal, hexagonal etc. cross sections, wherein in one implant all supply channels may have the same or different cross sections.

FIGS. 13 and 14 illustrate schematically two supply channel arrangements (sectioned again perpendicular to the supply channels), comprising supply channels having elongate, i.e. slot-shaped cross sections.

The arrangement shown in FIG. 13 is a staggered arrangement of supply channels 105 with slot-shaped cross sections having a width (smallest dimensions) in the range of 1 to 3 mm, and distances from each other in the given range of 2 to 6 mm, for example 3 to 5 mm. Dash-dotted lines indicate, as in FIG. 12, the volume of porous structure which can be supplied by each one of the supply channels 105.

The arrangement shown in FIG. 14 comprises supply channels 105 with slot-shaped and supply channels 105 with circular (or any other shape having a rotational symmetry) cross sections arranged in a regular pattern, and therewith constitutes an example of a supply channel arrangement comprising supply channels with differing cross sections. In any bone implant as above described, supply channels of other and possibly differing cross section shapes may be combined in varying numbers, wherein it is not necessary that the arrangement is as regular as shown in FIGS. 12 to 14.

FIG. 15 illustrates in a very schematic manner an exemplary bone implant, wherein for this implant, the distance between the two ingrowth surfaces 104 is in a range (20 to 40 mm) for which it is preferable to design the supply channels 105 to have enlarged mouth portions 125. Such enlarged mouth portions for example have dimensions in a range of 3 to 10 mm such that they are still able to be bridged by long-term bone growth without the necessity of bone growth enhancing material. Larger such enlarged mouth portions constituting critical size defects are not recommended because introduction of bone growth enhancing material is not really feasible. As shown in FIG. 15, the enlarged mouth portions 125 of the supply channels 105 are arranged alternatively in one and the other one of the ingrowth surfaces, which makes it possible to guarantee the given distances between the supply channels in the range of for example 3 to 5 mm. The axial length of the enlarged mouth portions 125 of the supply channels 105 may have a short axial length only, such maximizing spontaneous bone growth filling as much of the supply channels as possible.

All the above described features of bone implants, in particular regarding support frames and cross section shapes and supply channel arrangements are applicable also for embodiments comprising supply channels with enlarged mouth portions.

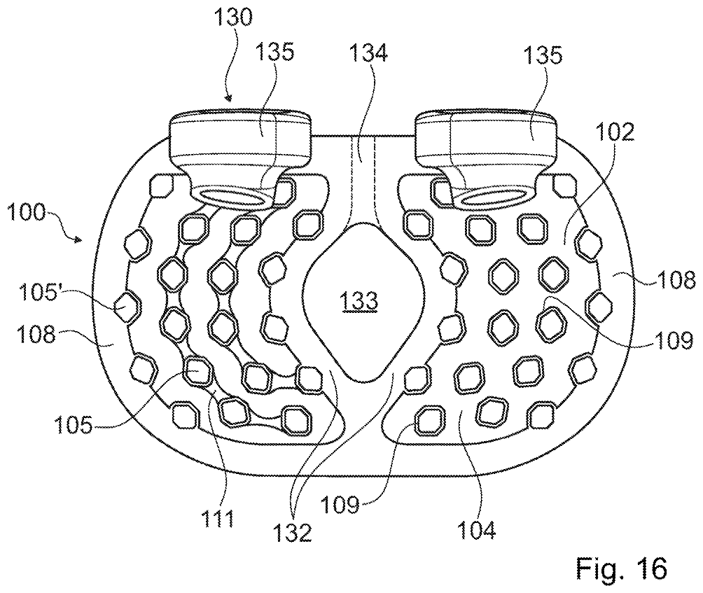

FIG. 16 is a top view (viewed against one of the ingrowth surfaces 104) of a further exemplary embodiment of the bone implant. The implant 100 is an interbody fusion device of the stand-alone type and comprises, as described above, a porous implant body 102 with supply channels 105 and a support frame with members 108 to be positioned between adjacent vertebral bodies. The interbody fusion device further comprises a bone plate 130 (retention means) and a plurality of bone anchors (not shown) suitable for fixating the bone plate 130 to lateral or posterior wall portions of the vertebral bodies. In the shown embodiment, the bone plate 130 is reduced to a plurality of lobes 135 each comprising a through opening for receiving one of the bone anchors. The porous implant body 102, the support frame members 108, the bone plate 130, and, if applicable, surface elements 109 or struts may be made of the same material, as one piece, which is for example manufactured in one single additive manufacturing process.

The porous implant body 102 comprises supply channels 105 as described above, mouths of the supply channels being at least partly surrounded by surface elements 109, possibly being connected to each other by connecting elements 111 (illustrated on left hand side of ingrowth surface). The support frame comprises edge members 108 and two central members 132, which extend through the porous implant body from the one ingrowth surface to the other one and which may or may not encircle a central cavity 133 suitable for being filled with bone growth enhancing graft material for which purpose this cavity 133 comprises a feed opening 134 connecting it with the proximal surface of the implant.

What is claimed is:

1. A fenestrated bone anchor suitable for being anchored in live bone tissue of a human or animal patient, wherein the bone anchor comprises a shaft with a proximal end, a distal end, a longitudinal axis and a circumferential surface extending from the proximal end to the distal end, wherein the shaft further comprises a longitudinal cavity extending in the direction of the axis from the proximal end towards the distal end and at least one lateral channel extending through a wall of the shaft from the axial cavity to the circumferential surface, the wall having a wall thickness and the lateral channel having a cross section with an axial length and a width smaller than the axial length, and wherein said width increases in a distal direction and, one of additionally or alternatively, said wall thickness increases gradually in at least selected ones of directions towards the lateral channel.

2. The bone anchor according to claim 1, comprising a plurality of lateral channels.

3. The bone anchor according to claim 2, wherein all lateral channels of the plurality of the lateral channels are arranged at a same first distance from the proximal end of the shaft and may be regularly spaced from each other around the circumferential surface.

4. The bone anchor according to claim 3, wherein the first distance is smaller than a second distance between the lateral channel and the distal end of the shaft.

5. The bone anchor according to claim 3 wherein the first distance is smaller than about one half of a total axial length of the shaft.

6. The bone anchor according to claim 1, wherein the wall thickness increases towards each one of the lateral channels and a cross section of the bone anchor or of the longitudinal cavity through the plurality of lateral channels has a lobed form having a lobe associated to each one of the channels.

7. The bone anchor according to claim 6, wherein in a proximal direction away from the plurality of lateral channels, the lobed form of the cross section of the bone anchor gradually transitions to a circular form.

8. The bone anchor according to claim 1 and further comprising a head and being suitable for fixating a bone plate relative to bone tissue, wherein the bone plate comprises a through opening adapted to the bone anchor.

9. The bone anchor according to claim 1 and further comprising retention structures arranged on the circumferential surface of the shaft.

10. The bone anchor according to claim 9, wherein the retention structures comprise at least one of a thread, circumferential ribs, sharp edges, teeth, surface roughness, undercut surface structures and an osseointegration enhancing surface coating.

11. The bone anchor according to claim 1, wherein at axial positions of the at least one lateral channel, the stiffness of the anchor against bending increases gradually towards proximally.

12. The bone anchor according to claim 1, wherein across a substantial portion an axial extension of the lateral channel the polar second moment of area of the bone anchor gradually increases towards proximally.

13. The bone anchor according to claim 1, wherein an average circumferential width of a proximal half of the at least one lateral channel is substantially smaller than an average circumferential width of a distal half of the lateral channel.

14. A surgical system comprising an implant with at least one through opening defining an opening axis and at least one bone anchor according to claim 1 and being suitable for fixating the implant relative to bone tissue of a human or animal patient, the bone anchor comprising a head and a shaft and an anchor axis, the through opening and the bone anchor being adapted to each other for the shaft to be able to pass through the through opening and the head to be retained by a proximal surface of the implant or within the through opening in a final position.

15. The system according to claim 14 wherein, for locking the anchor in said final position, the system further comprises at least one locking element being moveable between a relaxed position in which it protrudes from a general level of a surface portion of the anchor or the through opening and a resiliently tensioned position in which it protrudes less or not at all from said level, wherein the locking element is an integral part of the bone anchor or of the implant, constituting a part of said surface portion and of a bulk of the anchor or the implant situated underneath said surface portion, wherein a void in the surface portion delimits the locking element and further extends underneath the locking element or through said bulk.

16. The system according to claim 14, wherein the implant is a load bearing bone implant suitable for being implanted in a human or animal patient between surfaces of live bones or bone fragments, suitable for transmitting forces acting between the bones or bone fragments, and suitable for being integrated between the bones or bone fragments by bone growth after surgery, the bone implant comprising a porous implant body and a support frame, wherein the porous implant body comprises:
opposite ingrowth surfaces to be positioned against surfaces of the bones or bone fragments,
an open porosity constituting throughout the porous implant body a three-dimensional network of porosity channels of dimensions suitable for bone ingrowth, and
a plurality of supply channels, wherein each one of the supply channels has a mouth in at least one of the ingrowth surfaces and extends into or through the porous implant body substantially parallel to said forces, and wherein the supply channels have cross sections larger than the cross sections of the porosity channels and small enough for being bridgeable by spontaneous bone growth without additional bone growth enhancing material.

17. A surgical system comprising an implant with at least one through opening defining an opening axis and at least one bone anchor suitable for fixating the implant relative to bone tissue of a human or animal patient, the bone anchor comprising a head and a shaft and an anchor axis, the through opening and the bone anchor being adapted to each other for the shaft to be able to pass through the through opening and the head to be retained by a proximal surface of the implant or within the through opening in a final position, wherein, for locking the anchor in said final position, the system further comprises at least one locking element being moveable between a relaxed position in which it protrudes from a general level of a surface portion of the anchor or the through opening and a resiliently tensioned position in which it protrudes less or not at all from said level, wherein the locking element is an integral part of the bone anchor or of the implant, constituting a part of said surface portion and of a bulk of the anchor or the implant situated underneath said surface portion, wherein a void in the surface portion delimits the locking element and further extends underneath the locking element or through said bulk.

18. A load bearing bone implant suitable for being implanted in a human or animal patient between surfaces of live bones or bone fragments, suitable for transmitting forces acting between the bones or bone fragments, and suitable for being integrated between the bones or bone fragments by bone growth after surgery, the bone implant comprising a porous implant body and a support frame, wherein the porous implant body comprises:
two opposite ingrowth surfaces to be positioned against surfaces of the bones or bone fragments,
an open porosity constituting throughout the porous implant body a three-dimensional network of porosity channels of dimensions suitable for bone ingrowth, and
a plurality of supply channels, wherein each one of the supply channels has a length and a mouth in at least one of the ingrowth surfaces and extends towards the other ingrowth surface into the porous implant body, and wherein the supply channels have, at least over part of their length, cross sections larger than the cross sections of the porosity channels and small enough for being bridgeable by spontaneous bone growth without additional bone growth enhancing material.
